(19) United States
(12) Patent Application Publication
(10) Pub. No.: US 2022/0115424 A1
(43) Pub. Date: Apr. 14, 2022

(54) DETECTION DEVICE

(71) Applicant: Japan Display Inc., Tokyo (JP)

(72) Inventors: Tadayoshi KATSUTA, Tokyo (JP); Yoshitaka OZEKI, Tokyo (JP)

(21) Appl. No.: 17/494,918

(22) Filed: Oct. 6, 2021

(30) Foreign Application Priority Data
Oct. 8, 2020 (JP) 2020-170817

(51) Int. Cl. H01L 27/146; G06K 9/00

(52) U.S. Cl. CPC H01L 27/14605; G06K 9/0004; A61B 5/024; H01L 27/14623; H01L 27/14627; H01L 27/14607

(57) ABSTRACT

According to an aspect, a detection device includes: a substrate; a plurality of photodiodes arranged on the substrate; an insulating film that covers the photodiodes; and a conductive layer provided on the insulating film. Each photodiode includes a plurality of first regions, in each of which a p-type semiconductor layer, an i-type semiconductor layer, and an n-type semiconductor layer are stacked such that the p-type semiconductor layer is directly in contact with the i-type semiconductor layer and the i-type semiconductor layer is directly in contact with the n-type semiconductor layer. The i-type semiconductor layers in the first regions are separated from one another in a plan view. The n-type semiconductor layers in the first regions are separated from one another in the plan view. The conductive layer is coupled to each of the n-type semiconductor layers in the first regions through a contact hole provided in the insulating film.

### CROSS-REFERENCE TO RELATED APPLICATION

This application claims the benefit of priority from Japanese Patent Application No. 2020-170817 filed on Oct. 8, 2020, the entire contents of which are incorporated herein by reference.

### BACKGROUND

### 1. Technical Field

What is disclosed herein relates to a detection device.

### 2. Description of the Related Art

United States Patent Application Publication No. 2020/0089928 describes an optical imaging device that includes a light-blocking layer provided with an opening between a microlens and a photosensor. A positive-intrinsic-negative (PIN) photodiode is known as such a photosensor.

A detection device using such a PIN photodiode is required to be increased in detection sensitivity. For example, increasing a sensor area can increase a photocurrent of the PIN photodiode. Increasing the sensor area may, however, increase parasitic capacitance, and thus, reduce the detection sensitivity.

### SUMMARY

According to an aspect, a detection device includes: a substrate; a plurality of photodiodes arranged on the substrate; an insulating film that covers the photodiodes; and a conductive layer provided on the insulating film. Each of the photodiodes includes a plurality of first regions, in each of which a p-type semiconductor layer, an i-type semiconductor layer, and an n-type semiconductor layer are stacked such that the p-type semiconductor layer is directly in contact with the i-type semiconductor layer and the i-type semiconductor layer is directly in contact with the n-type semiconductor layer. The i-type semiconductor layers included in the first regions are provided so as to be separated from one another in a plan view, and the n-type semiconductor layers included in the first regions are provided so as to be separated from one another in the plan view. The conductive layer is coupled to each of the n-type semiconductor layers in the first regions through a contact hole provided in the insulating film.

### BRIEF DESCRIPTION OF THE DRAWINGS

FIG. 1A is a sectional view illustrating a schematic sectional configuration of a detection apparatus with an illumination device, the detection apparatus including a detection device according to an embodiment;

FIG. 1B is a sectional view illustrating a schematic sectional configuration of the detection apparatus with an illumination device, the detection apparatus including the detection device according to a first modification of the embodiment;

FIG. 1C is a sectional view illustrating a schematic sectional configuration of the detection apparatus with an illumination device, the detection apparatus including the detection device according to a second modification of the embodiment;

FIG. 1D is a sectional view illustrating a schematic sectional configuration of the detection apparatus with an illumination device, the detection apparatus including the detection device according to a third modification of the embodiment;

FIG. 2 is a plan view illustrating the detection device according to the embodiment;

FIG. 3 is a block diagram illustrating a configuration example of the detection device according to the embodiment;

FIG. 4 is a circuit diagram illustrating a detection element;

FIG. 5 is a timing waveform diagram illustrating an operation example of the detection element;

FIG. 6 is a plan view illustrating the detection element;

FIG. 7 is a VII-VII′ sectional view of FIG. 6;

FIG. 8 is a VIII-VIII′ sectional view of FIG. 6;

FIG. 9 is a sectional view illustrating a configuration example of an optical filter; and

FIG. 10 is a plan view illustrating a detection element according to a fourth modification of the embodiment.

### DETAILED DESCRIPTION

The following describes a mode (embodiment) for carrying out the present disclosure in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the disclosure. To further clarify the description, widths, thicknesses, shapes, and the like of various parts may be schematically illustrated in the drawings as compared with actual aspects thereof. However, they are merely examples, and interpretation of the present disclosure is not limited thereto. The same component as that described with reference to an already mentioned drawing is denoted by the same reference numeral through the disclosure and the drawings, and detailed description thereof may not be repeated where appropriate.

In the present specification and claims, in expressing an aspect of disposing another structure on or above a certain structure, a case of simply expressing “on” includes both a case of disposing the other structure immediately on the certain structure such that the other structure contacts the certain structure and a case of disposing the other structure above the certain structure with still another structure interposed therebetween, unless otherwise specified.

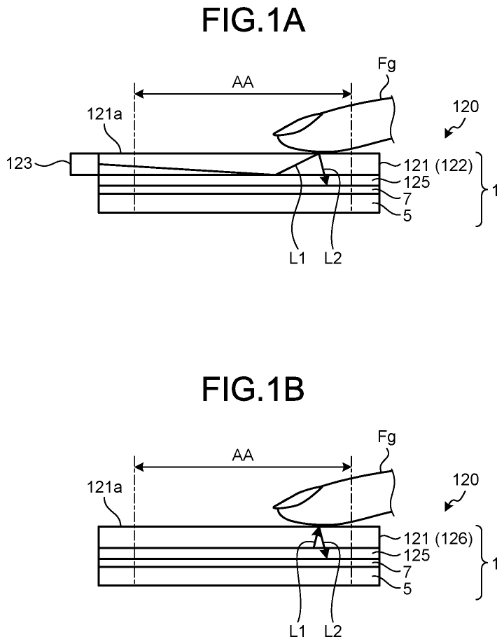

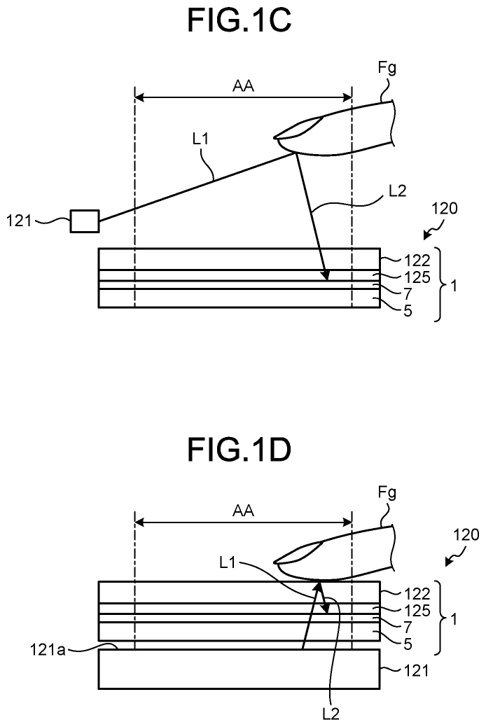

FIG. 1A is a sectional view illustrating a schematic sectional configuration of a detection apparatus with an illumination device, the detection apparatus including a detection device according to an embodiment. FIG. 1B is a sectional view illustrating a schematic sectional configuration of the detection apparatus with an illumination device, the detection apparatus including the detection device according to a first modification of the embodiment. FIG. 1C is a sectional view illustrating a schematic sectional configuration of the detection apparatus with an illumination device, the detection apparatus including the detection device according to a second modification of the embodiment. FIG. 1D is a sectional view illustrating a schematic sectional configuration of the detection apparatus with an illumination device, the detection apparatus including the detection device according to a third modification of the embodiment.

As illustrated in FIG. 1A, a detection apparatus 120 with an illumination device includes a detection device 1 and an illumination device 121. The detection device 1 includes a sensor substrate 5, an optical filter 7, an adhesive layer 125, and a cover member 122. That is, the sensor substrate 5, the optical filter 7, the adhesive layer 125, and the cover member 122 are stacked in the order as listed, in a direction orthogonal to a surface of the sensor substrate 5. The cover member 122 of the detection device 1 can be replaced with the illumination device 121, as will be described later. The adhesive layer 125 only needs to bond the optical filter 7 to the cover member 122. Hence, the detection device 1 may have a structure without the adhesive layer 125 in a region corresponding to a detection region AA. When the adhesive layer 125 is absent in the detection region AA, the detection device 1 has a structure in which the adhesive layer 125 bonds the cover member 122 to the optical filter 7 in a region corresponding to a peripheral region GA outside the detection region AA. The adhesive layer 125 provided in the detection region AA may be simply paraphrased as a protective layer for the optical filter 7.

As illustrated in FIG. 1A, the illumination device 121 may be, for example, what is called a side light-type front light that uses the cover member 122 as a light guide plate provided at a location corresponding to a detection region AA of the detection device 1, and that includes a plurality of light sources 123 arranged side by side at one end or both ends of the cover member 122. That is, the cover member 122 has a light-emitting surface 121a for emitting light, and serves as one component of the illumination device 121. The illumination device 121 emits light L1 from the light-emitting surface 121a of the cover member 122 toward a finger Fg serving as a detection target. For example, light-emitting diodes (LEDs) for emitting light in a predetermined color are used as the light sources.

As illustrated in FIG. 1B, the illumination device 121 may include light sources (such as LEDs) provided immediately below the detection region AA of the detection device 1, and the illumination device 121 including the light sources serves also as the cover member 122.

The illumination device 121 is not limited to the example of FIG. 1B. As illustrated in FIG. 1C, the illumination device 121 may be provided on a lateral side of or above the cover member 122, and may emit the light L1 to the finger Fg from the lateral side of or above the finger Fg.

Furthermore, as illustrated in FIG. 1D, the illumination device 121 may be what is called a direct-type backlight that includes light sources (such as LEDs) provided in the detection region of the detection device 1.

The light L1 emitted from the illumination device 121 is reflected as light L2 by the finger Fg serving as the detection target. The detection device 1 detects the light L2 reflected by the finger Fg (shading of the light L2 or an intensity of the reflected light) to detect asperities (such as a fingerprint) on the surface of the finger Fg. The detection device 1 may further detect the light L2 reflected inside the finger Fg to detect information on a living body in addition to detecting the fingerprint. Examples of the information on the living body include a blood vessel image of, for example, a vein, pulsation, and a pulse wave. The color of the light L1 from the illumination device 121 may be varied depending on the detection target.

The cover member 122 is a member for protecting the sensor substrate 5 and the optical filter 7, and covers the sensor substrate 5 and the optical filter 7. The illumination device 121 may have a structure to double as the cover member 122 as described above. In the structures illustrated in FIGS. 1C and 1D in which the cover member 122 is separate from the illumination device 121, the cover member 122 is, for example, a glass substrate. The cover member 122 is not limited to the glass substrate, and may be, for example, a resin substrate or may be constituted by a plurality of layers obtained by stacking these substrates. The cover member 122 need not be provided. In this case, the surface of the sensor substrate 5 and the optical filter 7 is provided with a protective layer of, for example, an insulating film, and the finger Fg contacts the protective layer of the detection device 1.

As illustrated in FIG. 1B, the detection apparatus 120 with an illumination device may be provided with a display panel 126 instead of the illumination device 121. The display panel 126 may be, for example, an organic electroluminescent (EL) diode (organic light-emitting diode (OLED)) display panel or an inorganic EL display (micro-LED or mini-LED). Alternatively, the display panel 126 may be a liquid crystal display (LCD) panel using liquid crystal elements as display elements or an electrophoretic display (EPD) panel using electrophoretic elements as the display elements. In this case, display light (light L1) emitted from the display panel 126 is reflected by the finger Fg, and the reflected light passes through the display panel 126 and reaches the optical filter 7. In view of this fact, the display panel 126 is preferably configured with a substrate or a multilayered film having light transmissivity. The fingerprint of the finger Fg and the information on the living body can be detected based on the light L2.

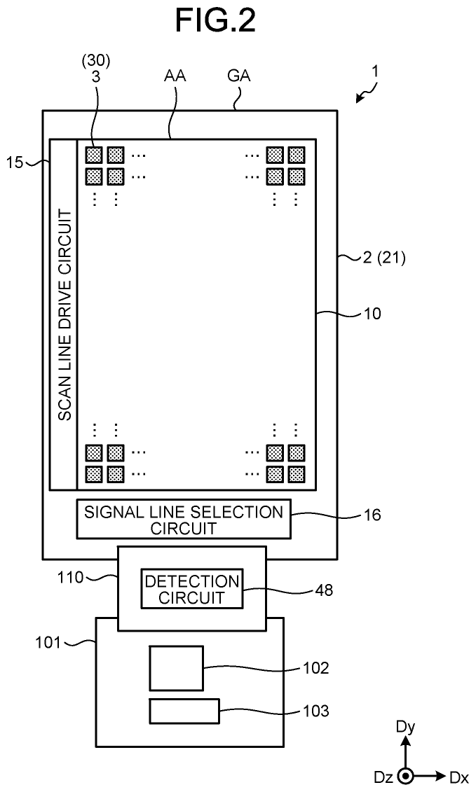

FIG. 2 is a plan view illustrating the detection device according to the embodiment. A first direction Dx illustrated in FIG. 2 and the subsequent drawings is one direction in a plane parallel to a substrate 21. A second direction Dy is a direction in the plane parallel to the substrate 21 and is a direction orthogonal to the first direction Dx. The second direction Dy may intersect the first direction Dx without being orthogonal thereto. A third direction Dz is a direction orthogonal to the first direction Dx and the second direction Dy and is a direction normal to the substrate 21.

As illustrated in FIG. 2, the detection device 1 includes an array substrate 2 (substrate 21), a sensor 10, a scan line drive circuit 15, a signal line selection circuit 16, a detection circuit 48, a control circuit 102, and a power supply circuit 103.

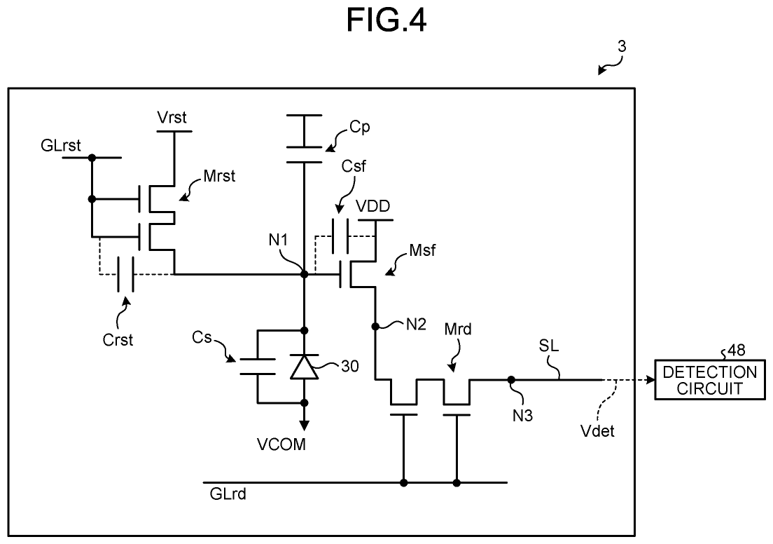

The substrate 21 is electrically coupled to a control substrate 101 through a wiring substrate 110. The wiring substrate 110 is, for example, a flexible printed circuit board or a rigid circuit board. The wiring substrate 110 is provided with the detection circuit 48. The control substrate 101 is provided with the control circuit 102 and the power supply circuit 103. The control circuit 102 is, for example, a field-programmable gate array (FPGA). The control circuit 102 supplies control signals to the sensor 10, the scan line drive circuit 15, and the signal line selection circuit 16 to control operations of the sensor 10. The power supply circuit 103 supplies voltage signals including, for example, a power supply potential VDD and a reference potential VCOM (refer to FIG. 4) to the sensor 10, the scan line drive circuit 15, and the signal line selection circuit 16. Although the present embodiment exemplifies the case of disposing the detection circuit 48 on the wiring substrate 110, the present disclosure is not limited to this case. The detection circuit 48 may be disposed on the substrate 21.

The substrate 21 has the detection region AA and the peripheral region GA. The detection region AA and the peripheral region GA extend in planar directions parallel to the substrate 21. Elements (detection elements 3) of the sensor 10 are provided in the detection region AA. The peripheral region GA is a region outside the detection region AA and is a region not provided with the elements (detection elements 3). That is, the peripheral region GA is a region between the outer circumference of the detection region AA and outer edges of the substrate 21. The scan line drive circuit 15 and the signal line selection circuit 16 are provided in the peripheral region GA. The scan line drive circuit 15 is provided in a region extending along the second direction Dy in the peripheral region GA. The signal line selection circuit 16 is provided in a region extending along the first direction Dx in the peripheral region GA, and is provided between the sensor 10 and the detection circuit 48.

Each of the detection elements 3 of the sensor 10 is a photosensor including a photodiode 30 as a sensor element. The photodiode 30 is a photoelectric conversion element, and outputs an electrical signal corresponding to light irradiating each of the photodiodes 30. More specifically, the photodiode 30 is a positive-intrinsic-negative (PIN) photodiode. The photodiode 30 may be paraphrased as an organic photodiode (OPD). The detection elements 3 are arranged in a matrix having a row-column configuration in the detection region AA. The photodiode 30 included in each of the detection elements 3 performs the detection in accordance with a gate drive signal (for example, a reset control signal RST or a read control signal RD) supplied from the scan line drive circuit 15. Each of the photodiodes 30 outputs the electrical signal corresponding to the light irradiating the photodiode 30 as a detection signal Vdet to the signal line selection circuit 16. The detection device 1 detects the information on the living body based on the detection signals Vdet received from the photodiodes 30.

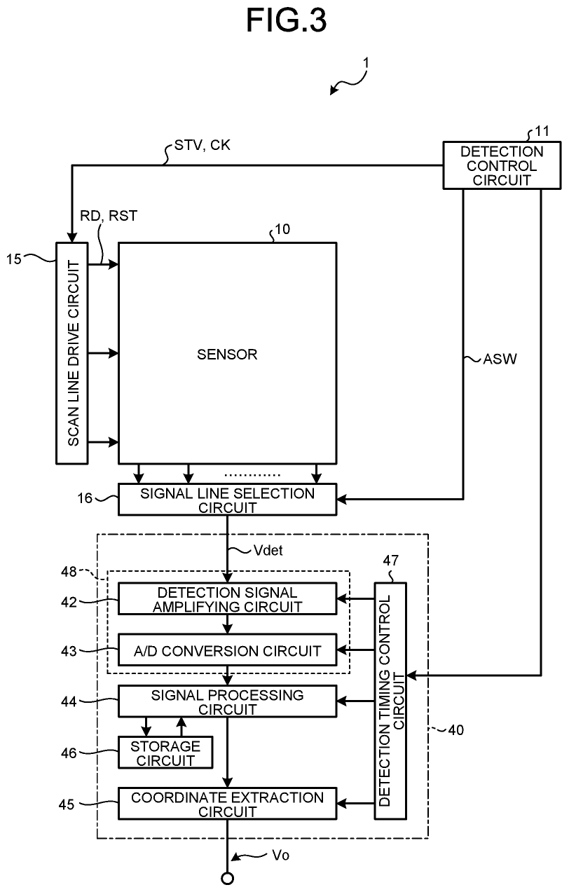

FIG. 3 is a block diagram illustrating a configuration example of the detection device according to the embodiment. As illustrated in FIG. 3, the detection device 1 further includes a detection control circuit 11 and a detector 40. One, some, or all functions of the detection control circuit 11 are included in the control circuit 102. One, some, or all functions of the detector 40 other than those of the detection circuit 48 are also included in the control circuit 102.

The detection control circuit 11 is a circuit that supplies control signals to the scan line drive circuit 15, the signal line selection circuit 16, and the detector 40 to control operations of these components. The detection control circuit 11 supplies various control signals including, for example, a start signal STV and a clock signal CK to the scan line drive circuit 15. The detection control circuit 11 also supplies various control signals including, for example, a selection signal ASW to the signal line selection circuit 16.

The scan line drive circuit 15 is a circuit that drives a plurality of scan lines (read control scan line GLrd and reset control scan line GLrst (refer to FIG. 4)) based on various control signals. The scan line drive circuit 15 sequentially or simultaneously selects the scan lines, and supplies the gate drive signal (for example, the reset control signal RST or the read control signal RD) to the selected scan lines. Through this operation, the scan line drive circuit 15 selects the photodiodes 30 coupled to the scan lines.

The signal line selection circuit 16 is a switching circuit that sequentially or simultaneously selects output signal lines SL (refer to FIG. 4). The signal line selection circuit 16 is, for example, a multiplexer. The signal line selection circuit 16 couples the selected output signal lines SL to the detection circuit 48 based on the selection signal ASW supplied from the detection control circuit 11. Through this operation, the signal line selection circuit 16 outputs the detection signal Vdet of the photodiode 30 to the detector 40.

The detector 40 includes the detection circuit 48, a signal processing circuit 44, a coordinate extraction circuit 45, a storage circuit 46, and a detection timing control circuit 47. The detection timing control circuit 47 performs control to cause the detection circuit 48, the signal processing circuit 44, and the coordinate extraction circuit 45 to operate in synchronization with one another based on a control signal supplied from the detection control circuit 11.

The detection circuit 48 is, for example, an analog front end (AFE) circuit. The detection circuit 48 is a signal processing circuit having functions of at least a detection signal amplifying circuit 42 and an analog-to-digital (A/D) conversion circuit 43. The detection signal amplifying circuit 42 is a circuit that amplifies the detection signal Vdet, and is, for example, an integration circuit. The A/D conversion circuit 43 converts an analog signal output from the detection signal amplifying circuit 42 into a digital signal.

The signal processing circuit 44 is a logic circuit that detects a predetermined physical quantity received by the sensor 10 based on output signals of the detection circuit 48. The signal processing circuit 44 can detect asperities on a surface of the finger Fg or a palm (fingerprint or palm print) based on the signals from the detection circuit 48 when the finger Fg is in contact with or in proximity to a detection surface. The signal processing circuit 44 may detect the information on the living body based on the signals from the detection circuit 48. Examples of the information on the living body include a blood vessel image of the finger Fg or the palm, a pulse wave, pulsation, and blood oxygen saturation.

The storage circuit 46 temporarily stores therein signals calculated by the signal processing circuit 44. The storage circuit 46 may be, for example, a random-access memory (RAM) or a register circuit.

The coordinate extraction circuit 45 is a logic circuit that obtains detected coordinates of the asperities on the surface of the finger Fg or the like when the contact or proximity of the finger Fg is detected by the signal processing circuit 44. The coordinate extraction circuit 45 is the logic circuit that also obtains detected coordinates of blood vessels of the finger Fg or the palm. The coordinate extraction circuit 45 combines the detection signals Vdet output from the respective detection elements 3 of the sensor 10 to generate two-dimensional information representing a shape of the asperities on the surface of the finger Fg or the like. The coordinate extraction circuit 45 may output the detection signals Vdet as sensor outputs Vo instead of calculating the detected coordinates.

The following describes a circuit configuration example of the detection device 1. FIG. 4 is a circuit diagram illustrating the detection element. As illustrated in FIG. 4, the detection element 3 includes the photodiode 30, a reset transistor Mrst, a read transistor Mrd, and a source follower transistor Msf. The reset transistor Mrst, the read transistor Mrd, and the source follower transistor Msf are provided correspondingly to each of the photodiodes 30. Each of the reset transistor Mrst, the read transistor Mrd, and the source follower transistor Msf is formed with an n-type thin-film transistor (TFT). However, each of the transistors is not limited thereto, and may be formed with a p-type TFT.

The reference potential VCOM is applied to an anode of the photodiode 30. A cathode of the photodiode 30 is coupled to a node N1. The node N1 is coupled to capacitance Cs, one of the source and the drain of the reset transistor Mrst, and the gate of the source follower transistor Msf. In addition, the node N1 has parasitic capacitance Cp and input capacitance Crst, Csf. When light enters the photodiode 30, a signal (electrical charge) output from the photodiode 30 is stored in the capacitance Cs.

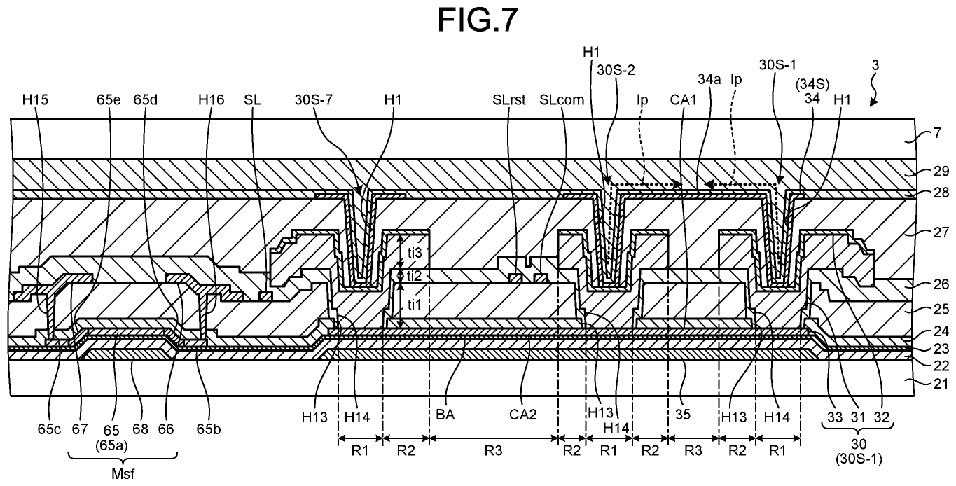

The capacitance Cs is, for example, capacitance generated between a p-type semiconductor layer 33 and an n-type semiconductor layer 32 of the photodiode 30 (refer to FIG. 7). The parasitic capacitance Cp is capacitance added to the capacitance Cs, and is also capacitance generated among various types of wiring and electrodes provided on the array substrate 2. The input capacitance Crst and the input capacitance Csf are capacitance of the reset transistor Mrst and capacitance of the source follower transistor Msf, respectively, as viewed from input sides thereof. More specifically, each of the input capacitance Crst and the input capacitance Csf is capacitance obtained by adding capacitance between the gate and the source to capacitance between the gate and the drain.

The gates of the reset transistor Mrst are coupled to the reset control scan line GLrst. One of the source and the drain of the reset transistor Mrst is supplied with a reset potential Vrst. When the reset transistor Mrst is turned on (into a conduction state) in response to the reset control signal RST, the potential of the node N1 is reset to the reset potential Vrst. The reference potential VCOM is lower than the reset potential Vrst, and the photodiode 30 is driven in a reverse bias state.

The source follower transistor Msf is coupled between a terminal supplied with the power supply potential VDD and the read transistor Mrd (node N2). The gate of the source follower transistor Msf is coupled to the node N1. The gate of the source follower transistor Msf is supplied with the signal (electrical charge) generated by the photodiode 30. This operation causes the source follower transistor Msf to output a voltage signal corresponding to the signal (electrical charge) generated by the photodiode 30 to the read transistor Mrd.

The read transistor Mrd is coupled between the source of the source follower transistor Msf (node N2) and the output signal line SL (node N3). The gates of the read transistor Mrd are coupled to the read control scan line GLrd. When the read transistor Mrd is turned on in response to the read control signal RD, the signal output from the source follower transistor Msf, that is, the voltage signal corresponding to the signal (electrical charge) generated by the photodiode 30 is output as the detection signal Vdet to the output signal line SL.

In the example illustrated in FIG. 4, the reset transistor Mrst and the read transistor Mrd each have what is called a double-gate structure configured by coupling two transistors in series. However, the reset transistor Mrst and the read transistor Mrd are not limited to this structure, and may have a single-gate structure, or a multi-gate structure including three or more transistors coupled in series. The circuit of the detection element 3 is not limited to the configuration including the three transistors of the reset transistor Mrst, the source follower transistor Msf, and the read transistor Mrd. The detection element 3 may include two transistors, or four or more transistors.

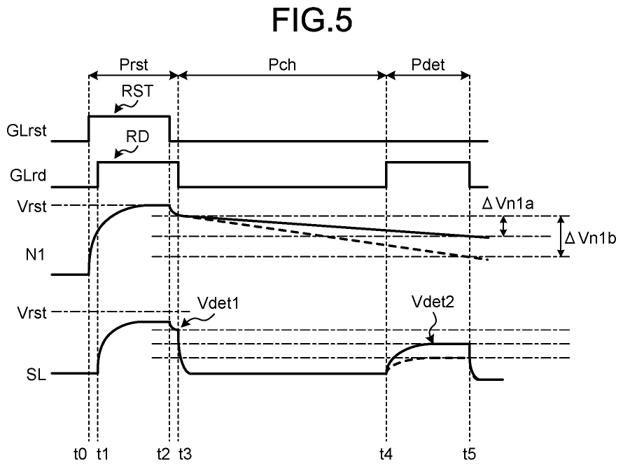

FIG. 5 is a timing waveform diagram illustrating an operation example of the detection element. As illustrated in FIG. 5, the detection element 3 performs the detection in the order of a reset period Prst, an exposure period Pch, and a reading period Pdet. The power supply circuit 103 supplies the reference potential VCOM to the anode of the photodiode 30 through the reset period Prst, the exposure period Pch, and the reading period Pdet.

At time to, the control circuit 102 sets the reset control signal RST to be supplied to the reset control scan line GLrst to HIGH (high-level voltage) to start the reset period Prst. In the reset period Prst, the reset transistor Mrst is tuned on (into the conductive state), and thus, the potential of the node N1 increases to the reset potential Vrst.

At time t1, the control circuit 102 sets the read control signal RD to be supplied to the read control scan line GLrd to HIGH (high-level voltage). As a result, the read transistor Mrd is turned on (into the conductive state).

At time t2, the control circuit 102 sets the reset control signal RST to LOW (low-level voltage), and thus, the reset period Prst ends. At time t2, the reset transistor Mrst is tuned off (into a nonconductive state). The signal corresponding to the light irradiating the photodiode 30 is stored to reduce the potential of the node N1 to (Vrst−ΔVn1). The term “ΔVn1” denotes a signal (voltage change amount) corresponding to the light irradiating the photodiode 30.

At time t3, the potential of the detection signal Vdet output from the output signal line SL reaches (Vrst−Vthsf−Vrdon). The term “Vthsf” denotes a threshold voltage Vthsf of the source follower transistor Msf. The term “Vrdon” denotes a voltage drop caused by an on-resistance of the read transistor Mrd.

At time t3, the control circuit 102 sets the read control signal RD to LOW (low-level voltage). As a result, the read transistor Mrd is turned off (into the nonconductive state). Thus, the potential of the node N2 is caused to be constant, and the potential of the detection signal Vdet output from the output signal line SL is also caused to be LOW (low-level voltage).

At time t4, the control circuit 102 sets the read control signal RD to HIGH (high-level voltage) again. As a result, the read transistor Mrd is turned on (into the conductive state). Thus, the exposure period Pch ends, and the reading period Pdet starts. The potential of a detection signal Vdet2 output during the reading period Pdet drops by the amount of the signal ΔVn1 from the potential of the detection signal Vdet1 obtained at time t3 to (Vrst−Vthsf−Vrdon−ΔVn1).

The detector 40 can detect the light irradiating the photodiode 30 based on the signal (ΔVn1) of the difference between the detection signal Vdet1 at time t3 and the detection signal Vdet2 at time t5. For example, a signal ΔVn1a illustrated in FIG. 5 is a signal (voltage change amount) generated when illuminance is low, and a signal ΔVn1b is a signal (voltage change amount) generated when the illuminance is high. The detection device 1 can detect the intensity of the light L2 for each of the detection elements 3 based on the difference between the signal ΔVn1a and the signal ΔVn1b. It is possible, by integrating the detection results of the individual detection elements 3, to detect, for example, the asperities of the fingerprint and the blood vessel image (vein pattern) of the finger applying force to the detection surface.

While FIG. 5 illustrates the operation example of one of the detection elements 3, the scan line drive circuit 15 can sequentially scan the reset control scan lines GLrst and the read control scan lines GLrd in a time division manner to cause the detection elements 3 in the entire detection region AA to perform the detection.

When capacitance Cn1 denotes a total of capacitance added to the photodiode 30, the capacitance Cn1 is represented by Expression (1) below, where the capacitance Cs, the parasitic capacitance Cp, the input capacitance Crst, and the input capacitance Csf are various types capacitance equivalently coupled to the cathode of the photodiode 30 (node N1) described above with reference to FIG. 4.

Cn1=Cs+Crst+Csf+Cp (1)

The signal ΔVn1 is represented by Expression (2) below, where ΔQ denotes an electrical charge stored during the exposure period Pch; Ip denotes a photocurrent that flows depending on the light irradiating the photodiode 30; and T denotes an exposure time (from time t3 to time t4).

ΔVn1=ΔQ/Cn1=(Ip×T)/Cn1 (2)

As represented by Expression (2), the signal ΔVn1 can be increased by reducing the capacitance Cn1. That is, the detection sensitivity of the detection device 1 is demonstrated to be increasable by reducing the capacitance Cn1 even when the same object to be detected is detected under the same detection condition.

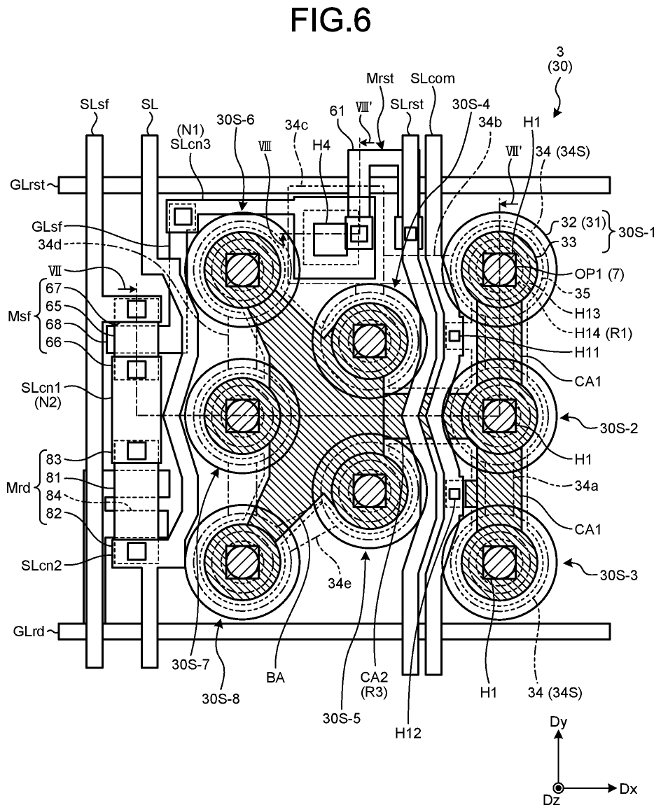

The following describes a planar configuration and a sectional configuration of the detection element 3. FIG. 6 is a plan view illustrating the detection element. As illustrated in FIG. 6, each of the detection elements 3 includes two scan lines (the read control scan line GLrd and the reset control scan line GLrst) and four signal lines (the output signal line SL, a power supply signal line SLsf, a reset signal line SLrst, and a reference signal line SLcom).

The read control scan lines GLrd and the reset control scan lines GLrst extend in the first direction Dx, and are arranged in the second direction Dy. The output signal lines SL, the power supply signal lines SLsf, the reset signal lines SLrst, and the reference signal lines SLcom extend in the second direction Dy, and are arranged in the first direction Dx.

The detection element 3 is defined as a region surrounded by the two scan lines (the read control scan line GLrd and the reset control scan line GLrst) and two signal lines (for example, two power supply signal lines SLsf of the adjacent detection elements 3).

As illustrated in FIG. 6, the photodiode 30 includes a plurality of partial photodiodes 30S-1, 30S-2, . . . , 30S-8. The partial photodiodes 30S-1, 30S-2, . . . , 30S-8 are arranged in a triangular lattice pattern.

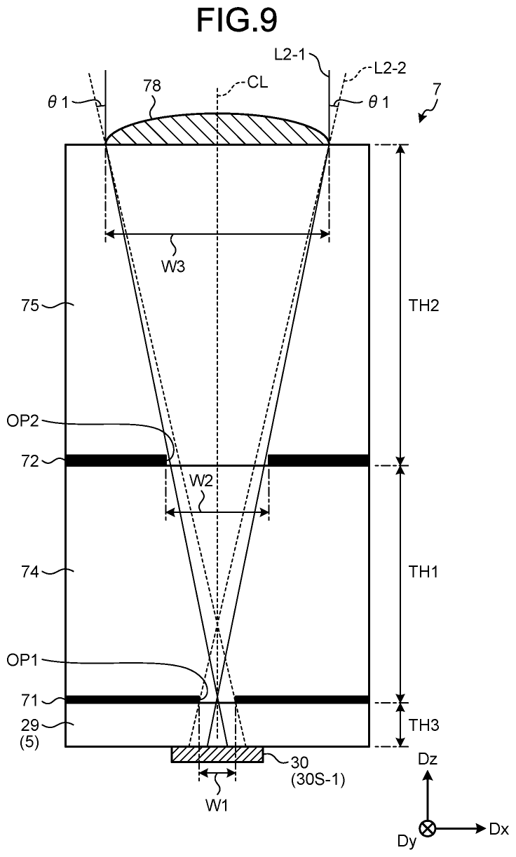

A lens 78 of the optical filter 7, a first opening OP1 of a first light-blocking layer 71, and a second opening OP2 of a second light-blocking layer 72, which are illustrated in FIG. 9, are provided so as to overlap each of the partial photodiodes 30S-1, 30S-2, . . . , 30S-8. For ease of viewing, FIG. 6 illustrates only the first opening OP1 among the components of the optical filter 7.

More specifically, the partial photodiodes 30S-1, 30S-2, and 30S-3 are arranged in the second direction Dy. The partial photodiodes 30S-4 and 30S-5 are arranged in the second direction Dy, and are adjacent to an element column made up of the partial photodiodes 30S-1, 30S-2, and 30S-3 in the first direction Dx. The partial photodiodes 30S-6, 30S-7, and 30S-8 are arranged in the second direction Dy, and are adjacent to an element column made up of the partial photodiodes 30S-4 and 30S-5 in the first direction Dx. The positions in the second direction Dy of the partial photodiodes 30S are arranged in a staggered manner between the adjacent element columns.

The light L2 is incident on the partial photodiodes 30S-1, 30S-2, . . . , 30S-8 through the optical filter 7. The partial photodiodes 30S-1, 30S-2, . . . , 30S-8 are electrically coupled together to serve as one photodiode 30. That is, signals output from the respective partial photodiodes 30S-1, 30S-2, . . . , 30S-8 are integrated into one detection signal Vdet to be output from the photodiode 30. In the following description, the partial photodiodes 30S-1, 30S-2, . . . , 30S-8 will be simply referred to as “partial photodiodes 30S” when need not be distinguished from one another.

Each of the partial photodiodes 30S includes an i-type semiconductor layer 31, the n-type semiconductor layer 32, and the p-type semiconductor layer 33. The i-type semiconductor layer 31 and the n-type semiconductor layer 32 are, for example, of amorphous silicon (a-Si). The p-type semiconductor layer 33 is, for example, of polysilicon (p-Si). The material of each of the semiconductor layers is not limited to those mentioned above and may be, for example, polysilicon or microcrystalline silicon.

The a-Si of the n-type semiconductor layer 32 is doped with impurities to form an n+ region. The p-Si of the p-type semiconductor layer 33 is doped with impurities to form a p+ region. The i-type semiconductor layer 31 is, for example, a non-doped intrinsic semiconductor and has lower conductivity than that of the n-type semiconductor layer 32 and the p-type semiconductor layer 33.

In FIG. 6, a dotted line indicates a first region R1 serving as an effective sensor region in which the p-type semiconductor layer 33 and the i-type semiconductor layer 31 (and the n-type semiconductor layer 32) overlap each other and are directly coupled together. Each of the partial photodiodes 30S includes at least the first region R1. In other words, a plurality of the first regions R1 are arranged in a triangular lattice pattern in a plan view. The first opening OP1 of the optical filter 7 is provided so as to overlap the first region R1.

Each of the partial photodiodes 30S is formed in a circular shape or a semi-circular shape in the plan view. The shape of the partial photodiode 30S is, however, not limited thereto, and may be, for example, a polygonal shape. The partial photodiodes 30S may have shapes different from one another.

The i-type semiconductor layers 31 and the n-type semiconductor layers 32 are arranged so as to be separated between the partial photodiodes 30S (first regions R1). The i-type semiconductor layers 31 and the n-type semiconductor layers 32 overlap the p-type semiconductor layers 33 of the first regions R1, and are provided so as to extend to regions around the first regions R1 not overlapping the p-type semiconductor layers 33. That is, the widths (diameters) of the i-type semiconductor layers 31 and the n-type semiconductor layers 32 are greater than the width (diameter) of the p-type semiconductor layers 33. In the present embodiment, the i-type semiconductor layers 31 and the n-type semiconductor layers 32 are provided concentrically with the p-type semiconductor layers 33.

The p-type semiconductor layers 33 of the partial photodiodes 30S-1, 30S-2, and 30S-3 arranged in the second direction Dy are electrically coupled together through joints CA1.

The p-type semiconductor layers 33 of the partial photodiodes 30S-4, 30S-5, 30S-6, 30S-7, and 30S-8 are electrically coupled together through a base BA. The base BA is formed in a substantially pentagonal shape and is provided, in the apex positions thereof, with the partial photodiodes 30S-4, 30S-5, 30S-6, 30S-7, and 30S-8.

The base BA coupled to the p-type semiconductor layers 33 of the partial photodiodes 30S-4, 30S-5, 30S-6, 30S-7, and 30S-8 is electrically coupled to the p-type semiconductor layers 33 of the partial photodiodes 30S-1, 30S-2, and 30S-3 through a joint CA2 passing below the reset signal line SLrst and the reference signal line SLcom along the first direction Dx. As a result, the partial photodiodes 30S constituting one photodiode 30 are electrically coupled together.

A lower conductive layer 35 is provided in a region overlapping the partial photodiodes 30S, the joints CA1 and CA2, and the base BA. Portions of the lower conductive layer 35 overlapping the respective partial photodiodes 30S are formed in a circular shape. However, the portions of the lower conductive layer 35 may have a different shape from that of the partial photodiode 30S. The lower conductive layer 35 only needs to be provided in portions overlapping at least the first regions R1. The lower conductive layer 35 is supplied with the reference potential VCOM that is the same as the potential of the p-type semiconductor layer 33, and thus can reduce the parasitic capacitance between the lower conductive layer 35 and the p-type semiconductor layer 33.

An upper conductive layer 34 (conductive layer) electrically couples the n-type semiconductor layers 32 of the partial photodiodes 30S to one another. Specifically, the upper conductive layer 34 includes main portions 34S overlapping the respective partial photodiodes 30S, and coupling portions 34a, 34b, 34c, 34d, and 34e for coupling the adjacent main portions 34S. The main portions 34S of the upper conductive layer 34 are electrically coupled to the n-type semiconductor layers 32 of the respective partial photodiodes 30S in positions overlapping the partial photodiodes 30S through contact holes H1 provided in an insulating film 27 (refer to FIG. 7). Each of the contact holes H1 has a quadrilateral shape (square shape) in the plan view as viewed in a direction orthogonal to the substrate 21. The shape of the contact hole H1 is not limited to this shape and may be another shape such as a circular shape in the plan view.

Each of the main portions 34S is provided concentrically with the n-type semiconductor layer 32 and the p-type semiconductor layer 33. The diameter of the main portion 34S is greater than the diameter of the p-type semiconductor layer 33 and is less than the diameter of the i-type semiconductor layer 31 and the diameter of the n-type semiconductor layer 32. The width of each of the coupling portions 34a, 34b, 34d, and 34e, more specifically, the width in a direction orthogonal to an extension direction of each of the coupling portions 34a, 34b, 34d, and 34e is less than the width (diameter) of the main portion 34S.

The coupling portions 34a of the upper conductive layer 34 extend in the second direction Dy so as to overlap the joint CA1, and couple together the main portions 34S overlapping the respective partial photodiodes 30S-1, 30S-2, and 30S-3. The coupling portion 34b of the upper conductive layer 34 extends in the first direction Dx, and one end of the coupling portion 34b is coupled to the main portion 34S overlapping the partial photodiode 30S-1. The other end of the coupling portion 34b of the upper conductive layer 34 is coupled to the coupling portion 34c. The coupling portion 34c of the upper conductive layer 34 is electrically coupled to the transistors (the reset transistor Mrst and the source follower transistor Msf (refer to FIG. 4)) through a contact hole H4.

The coupling portion 34c is coupled to the main portions 34S overlapping the respective partial photodiodes 30S-4 and 30S-6. The coupling portions 34d of the upper conductive layer 34 couple together the main portions 34S overlapping the respective partial photodiodes 30S-6, 30S-7, and 30S-8. The coupling portions 34d of the upper conductive layer 34 are arranged adjacent to the base BA in the first direction Dx and extend in the second direction Dy. Most parts of the coupling portions 34d of the upper conductive layer 34 are arranged so as not to overlap the base BA formed with the p-type semiconductor layers 33. The coupling portion 34e of the upper conductive layer 34 extends in an oblique direction intersecting the first direction Dx and the second direction Dy and couples together the main portions 34S overlapping the respective partial photodiodes 30S-8 and 30S-5. The coupling portion 34e of the upper conductive layer 34 extends along an outer edge of the base BA, and a most part thereof is disposed so as not to overlap the base BA.

The above-described configuration couples the upper conductive layer 34 (conductive layer) to the n-type semiconductor layers 32 of the first regions R1 through the contact holes H1 provided in the insulating film 27. The upper conductive layer 34 (conductive layer) couples one first region R1 to the next first region R1. The present embodiment can reduce the area of the upper conductive layer 34 overlapping the joints CA1 and CA2 and the base BA formed with the p-type semiconductor layers 33 as compared with a case where the upper conductive layer 34 is continuously formed so as to cover the partial photodiodes 30S, the joints CA1 and CA2, and the base BA. In the example illustrated in FIG. 6, the total number (six) of the coupling portions 34a, 34b, 34d, and 34e for coupling the main portions 34S is smaller than the numbers (eight) of the partial photodiodes 30S and the main portions 34S. This configuration can reduce the parasitic capacitance generated between the upper conductive layer 34 and the p-type semiconductor layers 33.

The upper conductive layer 34 may be provided in any manner, and, for example, the diameter of the main portion 34S of the upper conductive layer 34 may be greater than the diameters of the p-type semiconductor layer 33, the i-type semiconductor layer 31, and the n-type semiconductor layer 32. The number of the coupling portions 34a, 34b, 34d, and 34e may be equal to or greater than the number of the main portions 34S.

The reset transistor Mrst, the source follower transistor Msf, and the read transistor Mrd are provided in a region not overlapping the photodiode 30. The source follower transistor Msf and the read transistor Mrd are provided, for example, adjacent to the photodiode 30 in the first direction Dx. The reset transistor Mrst is provided adjacent to the partial photodiode 30S-4 in the second direction Dy and is provided between the partial photodiode 30S-1 and the partial photodiode 30S-6 in the first direction Dx.

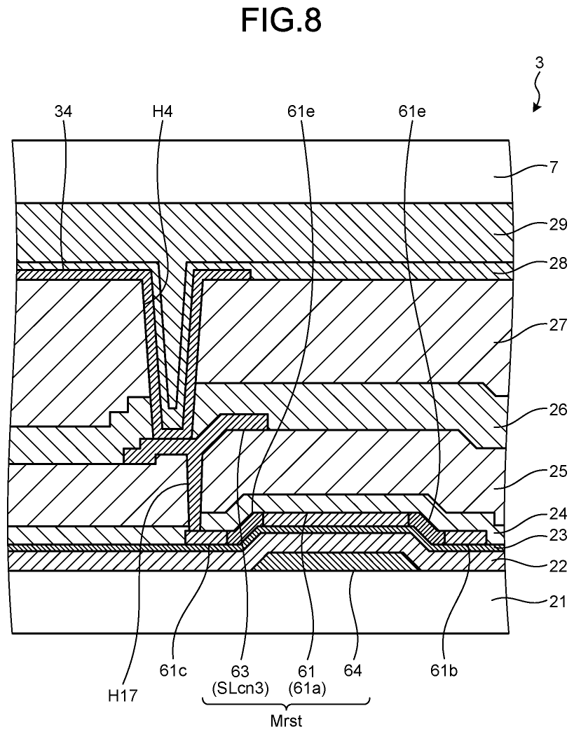

One end of a semiconductor layer 61 of the reset transistor Mrst is coupled to the reset signal line SLrst. The other end of the semiconductor layer 61 is coupled to coupling wiring SLcn3 (node N1) through a contact hole H17 (refer to FIG. 8). A portion of the reset signal line SLrst coupled to the semiconductor layer 61 serves as a source electrode, and a portion of the coupling wiring SLcn3 coupled to the semiconductor layer 61 serves as a drain electrode 63 (refer to FIG. 8). The semiconductor layer 61 is formed in a U-shape and intersects the reset control scan line GLrst at two locations. Channel regions are formed in portions of the semiconductor layer 61 overlapping the reset control scan line GLrst, and portions of the reset control scan line GLrst overlapping the semiconductor layer 61 serve as respective gate electrodes 64.

The source follower transistor Msf includes a semiconductor layer 65, a source electrode 66, a drain electrode 67, and a gate electrode 68. One end of the semiconductor layer 65 is coupled to the power supply signal line SLsf through a contact hole H15 (refer to FIG. 7). The other end of the semiconductor layer 65 is coupled to coupling wiring SLcn1 (node N2) through a contact hole H16 (refer to FIG. 7). A portion of the power supply signal line SLsf coupled to the semiconductor layer 65 serves as the drain electrode 67, and a portion of the coupling wiring SLcn1 coupled to the semiconductor layer 65 serves as the source electrode 66.

One end side of the gate electrode 68 extends in the first direction Dx and overlaps the semiconductor layer 65. The other end side of the gate electrode 68 extends in the second direction Dy and is electrically coupled to the coupling wiring SLcn3. This configuration electrically couples the reset transistor Mrst to the gate of the source follower transistor Msf through the coupling wiring SLcn3.

The read transistor Mrd includes a semiconductor layer 81, a source electrode 82, a drain electrode 83, and gate electrodes 84. One end of the semiconductor layer 81 is coupled to the coupling wiring SLcn1. The other end of the semiconductor layer 81 is coupled to coupling wiring SLcn2 branching in the first direction Dx from the output signal line SL. A portion of the coupling wiring SLcn1 coupled to the semiconductor layer 81 serves as the drain electrode 83, and a portion of the coupling wiring SLcn2 coupled to the semiconductor layer 81 serves as the source electrode 82. The two gate electrodes 84 are arranged in the second direction Dy and overlap the semiconductor layer 81. The two gate electrodes 84 are electrically coupled to the read control scan line GLrd through a branch that extends in the second direction Dy and overlaps the power supply signal line SLsf. The above-described configuration couples the source follower transistor Msf and the read transistor Mrd to the output signal line SL.

The output signal line SL is disposed between where the source follower transistor Msf and the read transistor Mrd are located and where the partial photodiodes 30S-6, 30S-7, and 30S-8 are located. The output signal line SL is provided in a zig-zag manner along the partial photodiodes 30S-6, 30S-7, and 30S-8.

The reset signal line SLrst and the reference signal line SLcom are arranged between the partial photodiodes 30S-1, 30S-2, 30S-3 and the partial photodiodes 30S-4, 30S-5. The reset signal line SLrst and the reference signal line SLcom are provided in a zig-zag manner along the partial photodiodes 30S and intersect the joint CA2. Since the partial photodiodes 30S-1, 30S-2, and 30S-3 are coupled to the partial photodiodes 30S-4 and 30S-5 through the joint CA2, the parasitic capacitance of the reset signal line SLrst and the reference signal line SLcom can be smaller than that in a configuration in which the base BA is provided so as to overlap the reset signal line SLrst and the reference signal line SLcom.

The reference signal line SLcom is electrically coupled to the lower conductive layer 35 through a contact hole H11. The reference signal line SLcom is electrically coupled to the joint CA2 through a contact hole H12. This configuration electrically couples the reference signal line SLcom to the p-type semiconductor layer 33 of each of the partial photodiodes 30S.

In the present embodiment, the partial photodiode 30S is provided for each of the first openings OP1 of the optical filter 7. This configuration can reduce portions of the semiconductor layers and wiring layers in a region not overlapping the first openings OP1 as compared with a configuration in which the photodiode 30 is formed of a solid film having, for example, a quadrilateral shape so as to cover the entire detection element 3 in the plan view. Thus, the parasitic capacitance of the photodiode 30 can be reduced. Since the multiple partial photodiodes 30S are provided, the degree of freedom of the layout of the transistors and the wiring can be increased, and thus, the transistors and the wiring can be provided so as not to overlap the photodiode 30. Consequently, in the present embodiment, the parasitic capacitance of the photodiode 30 can be smaller than that in a case of providing the photodiode 30 so as to overlap the transistors and the wiring.

In addition, the i-type semiconductor layers 31 and the n-type semiconductor layers 32 are arranged so as to be separated between the partial photodiodes 30S (first regions R1), and the n-type semiconductor layers 32 of the partial photodiodes 30S are coupled together by the upper conductive layer 34. This configuration can reduce the parasitic capacitance between the i-type and n-type semiconductor layers 31, 32 and the p-type semiconductor layer 33 as compared with a configuration in which the i-type and n-type semiconductor layers 31, 32 are continuously formed through the partial photodiodes 30S.

The planar structure of the photodiode 30 and the transistors illustrated in FIG. 6 is merely an example and can be changed as appropriate. For example, the number of the partial photodiodes 30S included in each of the photodiodes 30 may be seven or smaller, or nine of larger. The arrangement of the partial photodiodes 30S is not limited to the triangular lattice pattern. The partial photodiodes 30S may be arranged, for example, in a matrix.

FIG. 7 is a VII-VII′ sectional view of FIG. 6. FIG. 8 is a VIII-VIII′ sectional view of FIG. 6. FIG. 7 illustrates the sectional configuration of the partial photodiodes 30S-1, 30S-2, and 30S-7, and also the sectional configuration of the source follower transistor Msf included in the detection element 3. FIG. 8 illustrates the sectional configuration of the reset transistor Mrst. The read transistor Mrd (not illustrated in FIGS. 7 and 8) has the same sectional configuration as that of the source follower transistor Msf and the reset transistor Mrst.

As illustrated in FIG. 7, the substrate 21 is an insulating substrate, and is formed using, for example, a glass substrate of, for example, quartz or alkali-free glass, or a resin substrate of, for example, polyimide. The gate electrode 68 is provided on the substrate 21. Insulating films 22 and 23 are provided on the substrate 21 so as to cover the gate electrode 68. The insulating films 22 and 23 and insulating films 24, 25, and 26 are inorganic insulating films and are formed of, for example, a silicon oxide (SiO₂) or a silicon nitride (SiN).

The semiconductor layer 65 is provided on the insulating film 23. For example, polysilicon is used as the semiconductor layer 65. The semiconductor layer 65 is, however, not limited thereto, and may be formed of, for example, a microcrystalline oxide semiconductor, an amorphous oxide semiconductor, or low-temperature polycrystalline silicon (LTPS). The source follower transistor Msf has a bottom-gate structure in which the gate electrode 68 is provided on the lower side of the semiconductor layer 65. However, the source follower transistor Msf may have a top-gate structure in which the gate electrode 68 is provided on the upper side of the semiconductor layer 65, or a dual-gate structure in which the gate electrodes 68 are provided on the upper side and lower side of the semiconductor layer 65.

The semiconductor layer 65 has a channel region 65a, high-concentration impurity regions 65b and 65c, and low-concentration impurity regions 65d and 65e. The channel region 65a is, for example, a non-doped intrinsic semiconductor region or a low-impurity region, and has lower conductivity than that of the high-concentration impurity regions 65b and 65c and the low-concentration impurity regions 65d and 65e. The channel region 65a is provided in a region overlapping the gate electrode 68.

The insulating films 24 and 25 are provided on the insulating film 23 so as to cover the semiconductor layer 65. The source electrode 66 and the drain electrode 67 are provided on the insulating film 25. The source electrode 66 is coupled to the high-concentration impurity region 65b of the semiconductor layer 65 through the contact hole H16. The drain electrode 67 is coupled to the high-concentration impurity region 65c of the semiconductor layer 65 through the contact hole H15. The source electrode 66 and the drain electrode 67 are each formed of, for example, a multilayered film of Ti—Al—Ti or Ti—Al having a multilayered structure of titanium and aluminum.

As illustrated in FIG. 8, the reset transistor Mrst has the same sectional configuration as that of the source follower transistor Msf illustrated in FIG. 7. That is, the drain electrode 63 (coupling wiring SLcn3) is coupled to a high-concentration impurity region 61c of the semiconductor layer 61 through the contact hole H17. The insulating film 26 is provided on the insulating film 25 so as to cover the transistors including the reset transistor Mrst. The insulating film 27 is provided on the insulating film 26. The upper conductive layer 34 is provided on the insulating film 27. The upper conductive layer 34 is electrically coupled to the drain electrode 63 through the contact hole H4 provided through the insulating films 26 and 27.

Referring back to FIG. 7, the following describes the sectional configuration of the photodiode 30. While the partial photodiodes 30S-1, 30S-2, and 30S-7 are described with reference to FIG. 7, the description of the partial photodiodes 30S-1, 30S-2, and 30S-7 can also be applied to the other partial photodiodes 30S. As illustrated in FIG. 7, the lower conductive layer 35 is provided in the same layer as that of the gate electrode 68 on the substrate 21. The insulating films 22 and 23 are provided on the lower conductive layer 35. The photodiode 30 is provided on the insulating film 23. In other words, the lower conductive layer 35 is provided between the substrate 21 and the p-type semiconductor layer 33. The lower conductive layer 35 is formed of the same material as that of the gate electrode 68, and thereby serves as a light-blocking layer. Thus, the lower conductive layer 35 can restrain light from entering the photodiode 30 from the substrate 21 side.

The i-type semiconductor layer 31 is provided between the p-type semiconductor layer 33 and the n-type semiconductor layer 32 in the third direction Dz. In the present embodiment, the p-type semiconductor layer 33, the i-type semiconductor layer 31, and the n-type semiconductor layer 32 are stacked on the insulating film 23 in the order as listed.

Specifically, the p-type semiconductor layer 33 is provided in the same layer as the semiconductor layers 61 and 65 on the insulating film 23. The insulating films 24, 25, and 26 are provided so as to cover the p-type semiconductor layer 33. The insulating films 24 and 25 are provided with a contact hole H13 in a position overlapping the p-type semiconductor layer 33. The insulating film 26 is provided above the insulating film 25, and covers side surfaces of the insulating films 24 and 25 constituting an inner wall of the contact hole H13. The insulating film 26 is provided with a contact hole H14 in a position overlapping the p-type semiconductor layer 33.

The i-type semiconductor layer 31 is provided on the insulating film 26, and is coupled to the p-type semiconductor layer 33 through the contact hole H14 penetrating from the insulating film 24 to the insulating film 26. The n-type semiconductor layer 32 is provided on the i-type semiconductor layer 31.

In more detail, the photodiode 30 has the first regions R1, second regions R2 and third regions R3. The first regions R1 are provided correspondingly to the partial photodiodes 30S. In each of the first regions R1, the p-type semiconductor layer 33, the i-type semiconductor layer 31, and the n-type semiconductor layer 32 are stacked so as to be directly in contact with one another. In other words, the first region R1 is a region defined by a bottom surface of the contact hole H14.

The second regions R2 are provided between the first regions R1. The second regions R2 are provided around the first regions R1 in the plan view and are provided so as to be separated from one another on a partial photodiode 30S basis. In each of the second regions R2, at least the p-type semiconductor layer 33 and the i-type semiconductor layer 31 are stacked so as to be separated from each other in a direction orthogonal to the substrate 21 (in the third direction Dz). More specifically, the second region R2 includes the insulating films 24, 25, and 26 provided between the p-type semiconductor layer 33 and the i-type semiconductor layer 31. The second region R2 is, however, not limited thereto, and may include one or two layers of insulating films, or four or more layers of insulating films between the p-type semiconductor layer 33 and the i-type semiconductor layer 31.

In the second region R2, the thickness of the insulating films 24, 25, and 26 (the total thickness of a thickness ti1 of the insulating films 24 and 25 and a thickness ti2 of the insulating film 26) provided between the p-type semiconductor layer 33 and the i-type semiconductor layer 31 is greater than a thickness ti3 of the i-type semiconductor layer 31. The thickness ti1 of the insulating films 24 and 25 is greater than the thickness ti2 of the insulating film 26. The distance between the p-type semiconductor layer 33 and the n-type semiconductor layer 32 of the second region R2 is greater than the distance between the p-type semiconductor layer 33 and the n-type semiconductor layer 32 of the first region R1. The thickness relation between the i-type semiconductor layer 31 and the insulating films 24, 25, and 26 is not limited to the above-described relation, and a configuration can be employed in which the total thickness of the three layers of the insulating films 24, 25, and 26 is less than the thickness of the i-type semiconductor layer 31. In the second region R2, the insulating films 24, 25, and 26 having a predetermined thickness need to be present between the i-type semiconductor layer 31 (and/or the n-type semiconductor layer 32) and the p-type semiconductor layer 33. Various thicknesses can be employed as the thickness of the insulating films 24, 25, and 26.

The third regions R3 are provided between the adjacent second regions R2. More specifically, in each of the third regions R3, the p-type semiconductor layer 33 is provided, and the i-type semiconductor layer 31 and the n-type semiconductor layer 32 are provided so as not to overlap the p-type semiconductor layer 33. The distance between the p-type semiconductor layer 33 and the upper conductive layer 34 in the third region R3 (between the adjacent second regions R2) is greater than the distance between the p-type semiconductor layer 33 and the n-type semiconductor layer 32 in the second region R2. The third regions R3 include the joints CA1 and CA2 and the base BA. The partial photodiodes 30S-1, 30S-2, 30S-3 (FIG. 7 does not illustrate the partial photodiode 30S-3) are coupled together through the joints CA1 made with the p-type semiconductor layer 33 formed on the insulating film 23. In the same manner, the partial photodiode 30S-4 to 30S-8 (refer to FIG. 6) are coupled together through the base BA made with the p-type semiconductor layer 33 formed on the insulating film 23.

In the third region R3, the adjacent second regions R2 are coupled to each other at least through the p-type semiconductor layer 33. In addition, in the third region R3, the insulating films 24 and 25 are provided on the p-type semiconductor layer 33, and the reset signal line SLrst and the reference signal line SLcom are provided on the insulating films 24 and 25 provided on the p-type semiconductor layer 33. In other words, the i-type semiconductor layer 31 and the n-type semiconductor layer 32 are provided above the reset signal line SLrst and the reference signal line SLcom so as not to overlap the reset signal line SLrst and the reference signal line SLcom. Such a configuration can ensure insulation between each of the signal lines and the n-type semiconductor layer 32 as compared with a configuration in which the i-type semiconductor layer 31 and the n-type semiconductor layer 32 are provided so as to overlap the reset signal line SLrst and the reference signal line SLcom.

With the above-described configuration, the capacitance per unit area generated between the i-type semiconductor layer 31 and the p-type semiconductor layer 33 in the second regions R2 is smaller than the capacitance per unit area generated between the i-type semiconductor layer 31 and the p-type semiconductor layer 33 in the first regions R1. Consequently, the capacitance Cs of each of the photodiodes 30 (refer to FIG. 4) can be reduced as compared with the configuration in which the photodiode 30 is formed of a solid film having, for example, a quadrilateral shape so as to cover the entire detection element 3 in the plan view, that is, a configuration in which the i-type semiconductor layer 31 and the n-type semiconductor layer 32 of the second regions R2 are provided in the same layer as the first regions R1.

In the present embodiment, the third region R3 is provided between the adjacent first region R1 and the second region R2 and the adjacent first region R1 and the second region R2. The i-type semiconductor layers 31 and the n-type semiconductor layers 32 are provided so as to be separated between the partial photodiodes 30S, and the n-type semiconductor layers 32 of the adjacent partial photodiodes 30S are coupled together through the upper conductive layer 34. The capacitance per unit area generated between the upper conductive layer 34 and the p-type semiconductor layer 33 in the third region R3 is smaller than the capacitance per unit area generated between the i-type semiconductor layer 31 and the p-type semiconductor layer 33 in the second region R2. As a result, the capacitance Cs of each of the photodiodes 30 (refer to FIG. 4) can be reduced.

As a result, even under a detection condition where the same light is emitted and the same exposure time T is taken for exposure, the signal ΔVn1 can be increased as represented by Expression (2) given above, and thus, the detection sensitivity of the detection device 1 can be increased. The capacitance generated between the i-type semiconductor layer 31 and the p-type semiconductor layer 33 has been described above. However, in view of the fact that the i-type semiconductor layer 31 directly contacts the n-type semiconductor layer 32, and the p-type semiconductor layer faces the n-type semiconductor layer with the i-type semiconductor layer 31 interposed therebetween, the above description of the capacitance can naturally be replaced with a description of capacitance between the p-type semiconductor layer 33 and the n-type semiconductor layer 32.

The insulating film 27 is provided above the insulating film 26 so as to cover the photodiode 30. The insulating film 27 is provided so as to be directly in contact with the photodiode 30 and the insulating film 26. The insulating film 27 is formed of an organic material such as a photosensitive acrylic resin. The insulating film 27 is thicker than the insulating film 26. The thickness relation between these films may be reversed. The insulating film 27 has a better step coverage property than that of inorganic insulating materials, and is provided so as to cover side surfaces of the i-type semiconductor layer 31 and the n-type semiconductor layer 32.

The upper conductive layer 34 is provided above the insulating film 27. The upper conductive layer 34 is formed of, for example, a light-transmitting conductive material such as indium tin oxide (ITO). The main portion 34S and the coupling portions 34a, 34b, 34c, and 34d (FIG. 7 illustrates only the coupling portion 34a) of the upper conductive layer 34 are provided in the same layer on the insulating film 27. The upper conductive layer 34 is provided along a surface of the insulating film 27 and is coupled to the n-type semiconductor layer 32 through the contact hole H1 provided in the insulating film 27. With this configuration, signals (photocurrents Ip) output from the respective partial photodiodes 30S are integrated in the common upper conductive layer 34 and are output as one detection signal Vdet through the source follower transistor Msf and the read transistor Mrd (refer to FIG. 4).

The contact hole H1 is provided in a position overlapping the first region R1, and the n-type semiconductor layer 32 of the partial photodiode 30S is coupled to the upper conductive layer 34 (main portion 34S) on a bottom surface of the contact hole H1. However, since the upper conductive layer 34 only needs to be coupled to the n-type semiconductor layer 32 at any location in each of the partial photodiodes 30S, the contact hole H1 may be formed in a position offset from the center of the first region R1 of each of the partial photodiodes 30S. The upper conductive layer 34 is directly coupled to the n-type semiconductor layer 32 on the bottom surface of the contact hole H1, and the insulating film 27 is interposed between the upper conductive layer 34 and the n-type semiconductor layer 32 on the inner wall (side surface) of the contact hole H1. The present disclosure is, however, not limited to this configuration, and may employ a configuration in which the upper conductive layer 34 also directly contacts the n-type semiconductor layer 32 on the inner wall (side surface).

An insulating film 28 is provided on the insulating film 27 so as to cover the upper conductive layer 34. The insulating film 28 is an inorganic insulating film. The insulating film 28 is provided as a protective layer for restraining water from entering the photodiode 30.

A protective film 29 is provided on the insulating film 28. The protective film 29 is an organic protective film. The protective film 29 is formed so as to planarize a surface of the detection device 1.

In the present embodiment, the p-type semiconductor layer 33 and the lower conductive layer 35 of the photodiode 30 are provided in the same layers as those of the transistors. Therefore, the manufacturing process can be simpler than in a case where the photodiode 30 is formed in layers different from those of the transistors.

The sectional configuration of the photodiode 30 illustrated in FIG. 7 is merely an example. The sectional configuration is not limited to this example. For example, the photodiode 30 may be provided in layers different from those of the transistors. The stacking order of the p-type semiconductor layer 33, the i-type semiconductor layer 31, and the n-type semiconductor layer 32 is also not limited to that of FIG. 7. The stacking may be made in the order of the n-type semiconductor layer 32, the i-type semiconductor layer 31, and the p-type semiconductor layer 33.

The following describes a configuration example of the optical filter 7. FIG. 9 is a sectional view illustrating a configuration example of the optical filter. FIG. 9 illustrates a simplified configuration of the sensor substrate 5, and schematically illustrates the photodiode 30 (partial photodiode 30S-1) and the protective film 29 covering the photodiode 30.

The optical filter 7 is an optical element that transmits, toward the photodiode 30, a component of the light L2 reflected by an object to be detected such as the finger Fg that travels in the third direction Dz, and blocks components of the light L2 that travels in oblique directions. The optical filter 7 is also called collimator apertures or a collimator.

As illustrated in FIG. 9, the optical filter 7 includes the first light-blocking layer 71, the second light-blocking layer 72, a first light-transmitting resin layer 74, a second light-transmitting resin layer 75, and the lens 78. In the present embodiment, the first light-blocking layer 71, the first light-transmitting resin layer 74, the second light-blocking layer 72, the second light-transmitting resin layer 75, and the lens 78 are stacked on the protective film 29 in the order as listed.

The lenses 78 are respectively provided in regions overlapping the partial photodiodes 30S of the photodiodes 30. Each lens 78 is a convex lens. An optical axis CL of the lens 78 is provided in a direction parallel to the third direction Dz and intersects the partial photodiode 30S. The lens 78 is provided on the second light-transmitting resin layer 75 so as to be directly in contact therewith. In the present embodiment, no light-blocking layer or the like is provided on the second light-transmitting resin layer 75 between the adjacent lenses 78.

The first light-blocking layer 71 and the second light-blocking layer 72 are provided between the photodiode 30 and the lens 78 in the third direction Dz. The first light-blocking layer 71 is provided with the first opening OP1 in a region overlapping the photodiode 30. The second light-blocking layer 72 is provided with the second opening OP2 in a region overlapping the photodiode 30. The first opening OP1 and the second opening OP2 are formed in regions overlapping the optical axis CL.

The first light-blocking layer 71 is formed of, for example, a metal material such as molybdenum (Mo). This material allows the first light-blocking layer 71 to reflect the components of the light L2 traveling in the oblique directions other than the light L2 passing through the first opening OP1.

The second light-blocking layer 72 is formed of, for example, a resin material colored in black. With the above-described configuration, the second light-blocking layer 72 serves as a light-absorbing layer that absorbs the components of the light L2 traveling in the oblique directions other than the light L2 passing through the second opening OP2. For example, the second light-blocking layer 72 can absorb light reflected by the first light-blocking layer 71 and extraneous light incident from between the adjacent lenses 78.

In the present embodiment, the width decreases in the order of a width W3 of the lens 78 in the first direction Dx, a width W2 of the second opening OP2 in the first direction Dx, and a width W1 of the first opening OP1 in the first direction Dx. The width W1 of the first opening OP1 in the first direction Dx is less than the width of the partial photodiode 30S of the photodiode 30 in the first direction Dx.

A thickness TH2 of the second light-transmitting resin layer 75 illustrated in FIG. 9 is set to be greater than a thickness TH1 of the first light-transmitting resin layer 74. The thickness TH1 of the first light-transmitting resin layer 74 and the thickness TH2 of the second light-transmitting resin layer 75 are larger than a thickness TH3 of the protective film 29 of the sensor substrate 5.

With the above-described configuration, light L2-1 traveling in the third direction Dz among beams of the light L2 reflected by the object to be detected such as the finger Fg is condensed by the lens 78, and passes through the second opening OP2 and the first opening OP1 to enter the photodiode 30. Light L2-2 tilted by an angle 81 with respect to the third direction Dz also passes through the second opening OP2 and the first opening OP1 to enter the photodiode 30.

The optical filter 7 is integrally formed with the sensor substrate 5. That is, the first light-blocking layer 71 of the optical filter 7 is provided on the protective film 29 so as to be directly in contact therewith, and any member such as an adhesive layer is not provided between the first light-blocking layer 71 and the protective film 29. The optical filter 7 is directly formed as a film on the sensor substrate 5, and is formed by being subjected to a process such as patterning. Thus, the positional accuracy of the first opening OP1, the second opening OP2, and the lens 78 of the optical filter 7 with respect to the photodiode 30 can be improved as compared with the case of attaching the optical filter 7 as a separate component to the sensor substrate 5.

The configuration of the optical filter 7 illustrated in FIG. 9 is merely an example and can be changed as appropriate. For example, the optical filter 7 may be formed separately from the sensor substrate 5, and the relations between the widths W1, W2, and W3 and between the thicknesses TH1, TH2, and TH3 may also be changed as appropriate depending on required optical characteristics. The optical filter 7 is not limited to the configuration provided with the first light-blocking layer 71 and the second light-blocking layer 72, and may be provided with one layer of the first light-blocking layer 71.

### Modification

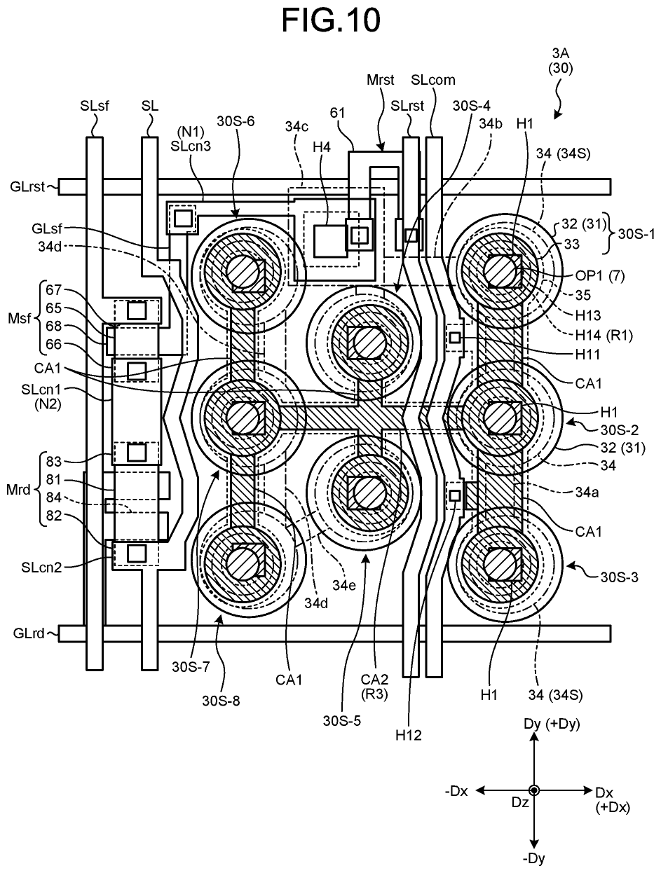

FIG. 10 is a plan view illustrating a detection element according to a fourth modification of the embodiment. In the following description, the same components as those described in the above-described embodiment are denoted by the same reference numerals, and the description thereof will not be repeated. In FIG. 10, for facilitating understanding of the description, a +Dx direction denotes the first direction Dx (rightward in FIG. 10), and a −Dx direction denotes the opposite direction of the first direction Dx (leftward in FIG. 10). Also, a +Dy direction denotes the second direction Dy (upward in FIG. 10), and a −Dy direction denotes the opposite direction of the second direction Dy (downward in FIG. 10).

As illustrated in FIG. 10, in a detection element 3A according to the fourth modification, the center positions of the i-type semiconductor layer 31 and the n-type semiconductor layer 32 are arranged so as to be offset from the center position of the p-type semiconductor layer 33 in each of the partial photodiodes 30S, unlike the above-described embodiment. In addition, the main portion 34S of the upper conductive layer 34 is disposed so as to overlap the i-type semiconductor layer 31 and the n-type semiconductor layer 32, and the center position of the main portion 34S of the upper conductive layer 34 is also disposed so as to be offset from the center position of the p-type semiconductor layer 33. Furthermore, along with the displacement of the i-type semiconductor layer 31, the n-type semiconductor layer 32, and the main portion 34S of the upper conductive layer 34, the position of the contact hole H1 provided in the insulating film 27 (refer to FIG. 7) is disposed so as to be offset from the center position of the first region R1.

In other words, in each of the partial photodiodes 30S (first regions R1), the distance, in the one direction of the first direction Dx (in the +Dx direction), from the outer circumference of the p-type semiconductor layer 33 to the outer circumference of the i-type semiconductor layer 31 and the n-type semiconductor layer 32 differs from the distance, in the other direction of the first direction Dx (in the −Dx direction), from the outer circumference of the p-type semiconductor layer 33 to the outer circumference of the i-type semiconductor layer 31 and the n-type semiconductor layer 32.

More specifically, in the partial photodiodes 30S-1, 30S-2, and 30S-3, the i-type semiconductor layer 31, the n-type semiconductor layer 32, and the main portion 34S of the upper conductive layer 34 are arranged so as to be offset in the +Dx direction with respect to the center position of the p-type semiconductor layer 33 of the first region R1. In the partial photodiodes 30S-4 and 30S-5, the i-type semiconductor layer 31, the n-type semiconductor layer 32, and the main portion 34S of the upper conductive layer 34 are arranged so as to be offset in the −Dx direction with respect to the center position of the p-type semiconductor layer 33 of the first region R1.

In the partial photodiode 30S-6, the i-type semiconductor layer 31, the n-type semiconductor layer 32, and the main portion 34S of the upper conductive layer 34 are arranged so as to be offset in the +Dx direction and the −Dy direction with respect to the center position of the p-type semiconductor layer 33 of the first region R1. In the partial photodiode 30S-7, the i-type semiconductor layer 31, the n-type semiconductor layer 32, and the main portion 34S of the upper conductive layer 34 are arranged so as to be offset in the +Dx direction with respect to the center position of the p-type semiconductor layer 33 of the first region R1. In the partial photodiode 30S-8, the i-type semiconductor layer 31, the n-type semiconductor layer 32, and the main portion 34S of the upper conductive layer 34 are arranged so as to be offset in the +Dx direction and the +Dy direction with respect to the center position of the p-type semiconductor layer 33 of the first region R1.

With this configuration, unlike the above-described embodiment, the i-type semiconductor layer 31 and the n-type semiconductor layer 32 of each of the partial photodiodes 30S-1, 30S-2, and 30S-3 can be restrained from overlapping the reference signal line SLcom. In the same manner, the i-type semiconductor layer 31 and the n-type semiconductor layer 32 of each of the partial photodiodes 30S-4 and 30S-5 can be restrained from overlapping the reset signal line SLrst. Furthermore, the i-type semiconductor layer 31 and the n-type semiconductor layer 32 of each of the partial photodiodes 30S-6, 30S-7, and 30S-8 can be restrained from overlapping the output signal line SL.

This configuration can ensure the insulation between the i-type and n-type semiconductor layers 31, 32 of each of the partial photodiodes 30S and the various signal lines. In another respect, this configuration can eliminate the necessity of a process, for example, to form the insulating film 27 (refer to FIG. 7) to be thicker, or to stack a plurality of the insulating films 27, in order to ensure the insulation between the i-type and n-type semiconductor layers 31, 32 and the various signal lines, and thus, can restrain the manufacturing cost of the detection device 1 from increasing.

The detection element 3A is provided with the joints CA1 and CA2 instead of the base BA for coupling together the p-type semiconductor layers 33 of the partial photodiodes 30S-4, 30S-5, 30S-6, 30S-7, and 30S-8. The p-type semiconductor layers 33 of the partial photodiodes 30S-4 and 30S-5 adjacent in the second direction Dy are coupled together through the joint CA1. The p-type semiconductor layers 33 of the partial photodiodes 30S-6, 30S-7, and 30S-8 arranged in the second direction Dy are coupled together through the joints CA1. The p-type semiconductor layers 33 of the partial photodiodes 30S-7 and 30S-2 adjacent in the first direction Dx are coupled together through the joint CA2 extending in the first direction Dx. The joint CA2 is provided so as to intersect the joint CA1 for coupling together the partial photodiodes 30S-4 and 30S-5, and passes below the reset signal line SLrst and the reference signal line SLcom along the first direction Dx.

The joints CA1 and CA2 for coupling together the partial photodiodes 30S-4, 30S-5, 30S-6, 30S-7, and 30S-8 have a smaller area than that of the base BA. As a result, when the positions of the i-type semiconductor layers 31, the n-type semiconductor layers 32, and the main portions 34S of the upper conductive layer 34 are displaced, the overlapping area of the i-type semiconductor layers 31, the n-type semiconductor layers 32, and the upper conductive layers 34 with the joints CA1 and CA2 formed with the p-type semiconductor layers 33 can be restrained from increasing. That is, the parasitic capacitance of the partial photodiodes 30S can be restrained from increasing.

As described above, each of the main portions 34S of the upper conductive layer 34 overlapping the partial photodiodes 30S-6, 30S-7, and 30S-8 is disposed so as to be offset in the +Dx direction with respect to the center position of the p-type semiconductor layer 33 of the first region R1. The first regions R1 of the partial photodiodes 30S-6, 30S-7, and 30S-8 are arranged in the second direction Dy, and the joints CA1 formed with the p-type semiconductor layers 33 extend in the second direction Dy to couple together the first regions R1. The coupling portions 34d of the upper conductive layer 34 are arranged so as to be adjacent to the joints CA1 in the first direction Dx (+Dx direction), and extend in the second direction Dy. This configuration can reduce the parasitic capacitance generated between the coupling portions 34d of the upper conductive layer 34 and the joints CA1.

The configuration of the detection element 3A illustrated in FIG. 10 can be combined with the above-described embodiment. For example, in the configuration provided with the base BA for coupling together the p-type semiconductor layers 33 of the partial photodiodes 30S-4, 30S-5, 30S-6, 30S-7, and 30S-8, the i-type semiconductor layers 31 and the n-type semiconductor layers 32 of the partial photodiodes 30S and the main portions 34S of the upper conductive layer 34 may be arranged so as to be offset. Alternatively, in the configuration in which the i-type semiconductor layers 31 and the n-type semiconductor layers 32 of the partial photodiodes 30S and the main portions 34S of the upper conductive layer 34 are arranged concentrically with the p-type semiconductor layers 33, the joints CA1 and CA2 for coupling together the p-type semiconductor layers 33 of the partial photodiodes 30S-4, 30S-5, 30S-6, 30S-7, and 30S-8 may be arranged instead of the base BA.

The present disclosure is not limited to the case of offsetting all of the i-type semiconductor layers 31 and the n-type semiconductor layers 32 of the partial photodiodes 30S and the main portions 34S of the upper conductive layer 34, and may employ a configuration of offsetting some of the i-type semiconductor layers 31 and the n-type semiconductor layers 32 of the partial photodiodes 30S and the main portions 34S of the upper conductive layer 34. The offset direction and the offset amount of each of the i-type semiconductor layers 31 and the n-type semiconductor layers 32 of the partial photodiodes 30S and the main portions 34S of the upper conductive layer 34 can be changed as appropriate depending on the positional relation with the signal lines. The arrangement of the signal lines is only an example and can be changed as appropriate. The arrangement of the transistors can also be changed depending on the number and arrangement of the partial photodiodes 30S.

While the preferred embodiment of the present disclosure has been described above, the present disclosure is not limited to the embodiment described above. The content disclosed in the embodiment is merely exemplary, and can be variously changed within the scope not departing from the gist of the present disclosure. Any modification appropriately made within the scope not departing from the gist of the present disclosure also naturally belongs to the technical scope of the present disclosure. At least one of various omissions, replacements, and modifications of the components can be made without departing from the gist of the embodiment and the modifications thereof described above.

What is claimed is:

1. A detection device comprising:
a substrate;
a plurality of photodiodes arranged on the substrate;
an insulating film that covers the photodiodes; and
a conductive layer provided on the insulating film, wherein
each of the photodiodes comprises a plurality of first regions, in each of which a p-type semiconductor layer, an i-type semiconductor layer, and an n-type semiconductor layer are stacked such that the p-type semiconductor layer is directly in contact with the i-type semiconductor layer and the i-type semiconductor layer is directly in contact with the n-type semiconductor layer,
the i-type semiconductor layers included in the first regions are provided so as to be separated from one another in a plan view, and the n-type semiconductor layers included in the first regions are provided so as to be separated from one another in the plan view, and
the conductive layer is coupled to each of the n-type semiconductor layers in the first regions through a contact hole provided in the insulating film.

2. The detection device according to claim 1, wherein the conductive layer comprises a plurality of main portions provided so as to overlap the respective first regions, and coupling portions that couple together the adjacent main portions.

3. The detection device according to claim 2, wherein
the adjacent first regions are coupled together through the p-type semiconductor layers, and
at least one of the coupling portions is disposed so as not to overlap the p-type semiconductor layer that couples together the adjacent first regions.

4. The detection device according to claim 2, wherein the coupling portions have widths less than widths of the main portions.

5. The detection device according to claim 1, wherein each of the first regions is surrounded second regions in each of which the p-type semiconductor layer, the i-type semiconductor layer, and the n-type semiconductor layer are stacked such that at least the p-type semiconductor layer and the i-type semiconductor layer face so as to be separated from each other in a thickness direction and the i-type semiconductor layer is directly in contact with the n-type semiconductor layer.

6. The detection device according to claim 5, wherein, when viewed in the thickness direction, a distance between the p-type semiconductor layer and the n-type semiconductor layer in each of the second regions is greater than a distance between the p-type semiconductor layer and the n-type semiconductor layer in each of the first regions.

7. The detection device according to claim 5, wherein, when viewed in the thickness direction, a distance between the p-type semiconductor layer and the conductive layer in a region between the adjacent second regions is greater than a distance between the p-type semiconductor layer and the n-type semiconductor layer in each of the second regions.

8. The detection device according to claim 1, wherein
diameters of the i-type semiconductor layer and the n-type semiconductor layer are set to be greater than a diameter of the p-type semiconductor layer, and
in each of the first regions, a distance, in a first direction parallel to the substrate, from an outer circumference of the p-type semiconductor layer to an outer circumference of the i-type semiconductor layer and the n-type semiconductor layer differs from a distance, in an opposite direction of the first direction, from the outer circumference of the p-type semiconductor layer to the outer circumference of the i-type semiconductor layer and the n-type semiconductor layer.

9. The detection device according to claim 8, wherein
the first regions are arranged in a second direction intersecting the first direction,
the detection device comprises a joint including the p-type semiconductor layer and extending in the second direction to couple together the first regions, and
the conductive layer is disposed so as to be adjacent to the joint in the first direction and extends in the second direction.

10. The detection device according to claim 5, wherein center positions of the i-type semiconductor layer and the n-type semiconductor layer included in the first region and the second region are arranged so as to be offset from a center position of the p-type semiconductor layer included in the first region, in a plan view.

11. The detection device according to claim 1, comprising transistors coupled to the photodiodes, wherein
the p-type semiconductor layer is provided in the same layer as a semiconductor layer of each of the transistors.

12. The detection device according to claim 1, wherein the first regions are arranged in a triangular lattice pattern in a plan view.

13. The detection device according to claim 1 comprising a optical filter overlapping the photodiodes, wherein
the optical filter has openings in positions overlapping the first regions.
